(19) United States
(12) Patent Application Publication
(10) Pub. No.: US 2020/0253473 A1
(43) Pub. Date: Aug. 13, 2020

(54) FLEXIBLE PRINTED CIRCUIT BOARD MODULE FOR SMART BAND

(71) Applicant: Korea Electronics Technology Institute, Seongnam-si (KR)

(72) Inventors: Joon Shik PARK, Gunpo-si (KR); Dong Sun KIM, Seongnam-si (KR); Tae Ho HWANG, Seoul (KR)

(73) Assignee: Korea Electronics Technology Institute, Seongnam-si (KR)

(21) Appl. No.: 15/776,264

(22) PCT Filed: Sep. 29, 2016
(86) PCT No.: PCT/KR2016/010905

§ 371 Date: May 15, 2018

(30) Foreign Application Priority Data
Nov. 19, 2015 (KR) 10-2015-0162190

(51) Int. Cl. A61B 5/00; A44C 5/00; H04Q 9/00; A61B 5/0255; A61B 5/11; A61B 5/16

(52) U.S. Cl. CPC A61B 5/002; A44C 5/0007; H04Q 9/00; A61B 5/0255; A61B 5/1118; A61B 5/02438; A61B 5/443; A61B 5/165; H04Q 2209/40; A61B 2560/0214; A61B 5/681

(57) ABSTRACT

The present invention relates to a flexible printed circuit board (FPCB) module for a smart band capable of a biometric signal measurement, the FPCB module comprising: a micro battery for supplying power; biometric signal detection sensors for detecting at least each of skin humidity, a body temperature, and a pulse signal; a motion detection sensor for detecting a dynamic motion signal; a signal processing unit configured to amplify and digitally convert signals detected by each of the sensors to output the signals as sensor detection signals; and a wireless communication unit configured to process and transmit the digitally converted sensor detection signals according to a wireless communication standard, wherein the micro battery, the biometric signal detection sensors, the motion detection sensor, the signal processing unit, and the wireless communication unit are mounted on a flexible printed circuit board and are interconnected through electrical wiring.

### TECHNICAL FIELD

The present invention relates to a smart band that can measure biometric signals and, more particularly, to a flexible printed circuit board (FPCB) module for a smart band.

### BACKGROUND ART

According to a recently released report, as a proportion of elderly people older than the age of 65 is expected to become about 25% of the total population by 2030, one fourth of the population is expected to be old persons. It is noted that interest in health also increases as a living standard becomes higher and life expectancy is extended. An increasing interest in health means that the use frequency of goods and services related to the health care industry increases.

Products related to the health care industry have become widespread already in our everyday real life. For example, personal body-shape diagnostic and calibration type programs, etc. are mounted in an electronic digital thermometer, a pulsimeter, a blood pressure gauge, a low-frequency therapeutic apparatus, a personal blood glucose meter, and various types of fitness equipment.

However, these healthcare related products are generally distributed as an independent and single product; and, because even a complex product having several mixed functions does not support a communication function, there is a limitation in terms of receiving a telemedicine service and there is a drawback that portability is not easy.

Therefore, when developing a healthcare related product having several mixed functions, if it is possible that such a healthcare related product can be easily carried and conveniently used, and can share information with a so-called expert group such as a medical team or a health trainer, or a body-shape manager located at a remote location, by using a wireless communication technology, it is expected that such a healthcare related product will occupy in advance a promising healthcare market.

When considering that there has been a great increase in interest in wearable computers, appearing currently as a hot issue, if it is possible that incorporating healthcare related products having the foregoing several functions into a smart watch or a smart band which is currently at an early distribution stage, it is expected that the new product will occupy in advance an initial wearable device market and contribute to revitalizing a healthcare market.

Further, a smart watch or a smart band, as products that meet such an expectation, has been on sale already. Generally, the smart band on sale has a pulse sensor and an acceleration sensor therein, and thus is used in a service for measuring a heart rate and calculating and providing a quantity of exercise from the measured heart rate. That is, a conventional smart band has a limitation in terms of providing a wearer with a service that can monitor the wearer's feeling and emotion by measuring various biometric signals.

Further, because sensors used in an introduced smart band are rigid as a whole, the feeling while wearing the smart band is not good; and, because a close contact state of the smart band with the skin of a wearer is not maintained, there are disadvantages that a biometric signal cannot be measured or a large measurement error occurs, during a dynamic state (walking, running, and exercising).

### PRIOR ART DOCUMENT

[Patent Document]

[Patent Document 1] Korean Laid-Open Patent Publication No. 10-2013-0033752

### Technical Problem

The present invention has been made to solve the above problems and is intended to provide an FPCB module for a smart band that can support monitoring even an emotion or a feeling such as stress by measuring more biometric signals in addition to a pulse.

The present invention further provides an FPCB module for a smart band that enables a wearer to measure normally biometric signals even the wearer is in a dynamically moving condition and that enables the measurement of biometric signals that can monitor an emotion or a feeling.

### Solution to Problem

In accordance with an aspect of the present invention, a flexible printed circuit board (FPCB) module for a smart band comprises

a micro battery for supplying power;

biometric signal detection sensors for detecting at least each of skin humidity, a body temperature, and a pulse signal;

a motion detection sensor for detecting a dynamic movement signal;

a signal processing unit configured to amplify and digitally convert signals detected by each of the sensors to output the signals as sensor detection signals; and

a wireless communication unit configured to process and transmit the digitally converted sensor detection signals according to a wireless communication standard,

wherein the micro battery, the biometric signal detection sensors, the motion detection sensor, the signal processing unit, and the wireless communication unit are mounted on the FPCB to be interconnected through electrical wiring.

Further, the biometric signal detection sensors are integrated into a flexible sensor module on the FPCB, and the micro battery, the motion detection sensor, the signal processing unit, and the wireless communication unit are integrated into a signal processing module on the FPCB so that the flexible sensor module and the signal processing module are physically separated.

In accordance with another aspect of the present invention, a flexible printed circuit board (FPCB) module for a smart band comprises

a flexible sensor module in which biometric signal detection sensors for detecting at least each of skin humidity, a body temperature, and pulse signals are mounted on a first FPCB; and

a signal processing module in which a movement detection sensor for detecting a dynamic movement signal, a signal processing unit configured to amplify and digitally convert signals detected by each of the sensors to output the signals as sensor detection signals, and a wireless communication unit configured to process and transmit the digitally converted sensor detection signals according to a wireless communication standard are mounted on a second FPCB,

wherein the flexible sensor module and the signal processing module are interconnected through conductive fiber or flexible wiring.

### Advantageous Effects

According to the above problem solving means, because an FPCB module for a smart band according to an embodiment of the present invention can detect a body temperature and skin moisture together as a biometric signal, as well as detect a pulse, a smart band wearer can monitor an emotional change of oneself, and thus, can cope wisely with a sudden dangerous situation.

Further, because biometric signal detection sensors are mounted on an FPCB, the sensors come into a close contact with a body of a wearer, and thus, even if the wearer walks or runs, can measure biometric signals such as a heart rate normally, and can minimize an attachment feeling of foreign matters due to good adhesion when the smart band is worn.

In some cases, by mounting and separating a signal processing module and a flexible sensor module in different FPCBs, flexibility, elasticity, and wearing convenience can be provided together when the smart band is worn.

### BRIEF DESCRIPTION OF DRAWINGS

FIG. 1 is a block diagram illustrating a configuration of an FPCB module for a smart band according to an embodiment of the present invention.

FIG. 2 is a block diagram illustrating a configuration of an FPCB module for a smart band according to another embodiment of the present invention.

FIG. 3 illustrates a manufactured implementation of an FPCB module for a smart band according to an embodiment of the present invention.

### MODE FOR THE INVENTION

Hereinafter, embodiments of the present invention will be described in detail with reference to the accompanying drawings. Detailed descriptions of well-known functions and structures incorporated herein may be omitted to avoid obscuring the subject matter of the present invention.

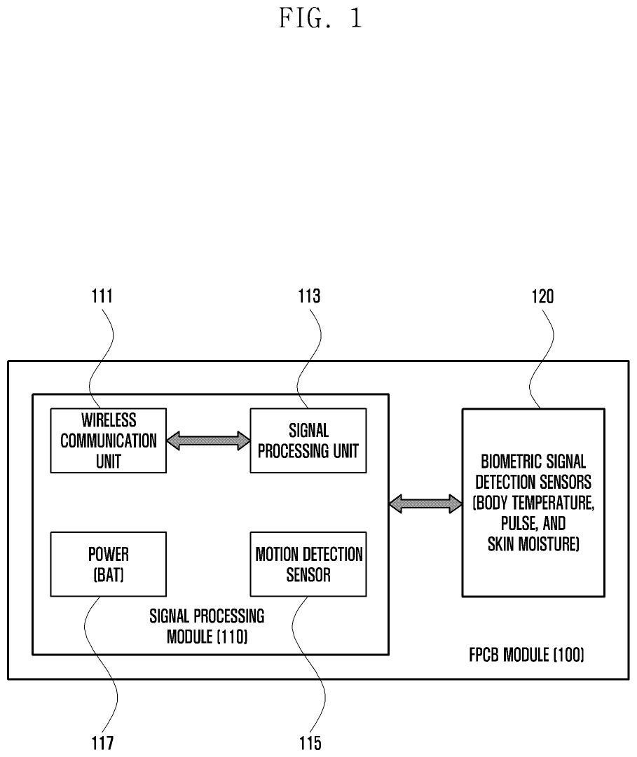

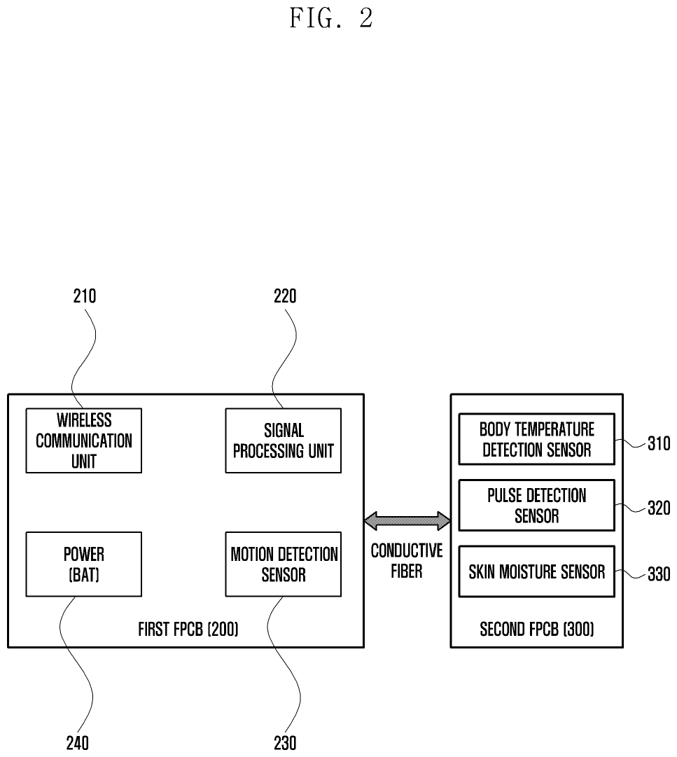

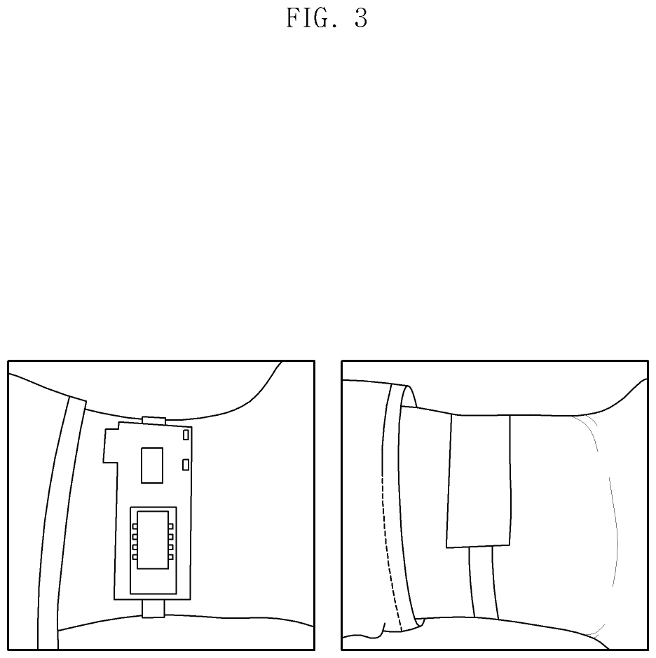

FIG. 1 is a block diagram illustrating a configuration of an FPCB module for a smart band according to an embodiment of the present invention, and FIG. 2 is a block diagram illustrating a configuration of an FPCB module for a smart band according to another embodiment of the present invention. FIG. 3 illustrates a manufactured implementation of an FPCB module for a smart band according to an embodiment of the present invention.

With reference to FIG. 1, an FPCB module for a smart band according to an embodiment of the present invention comprises

a micro battery 117 for supplying operation power to each component element constituting an FPCB module for a smart band that can measure biometric signals;

biometric signal detection sensors 120 for detecting at least each signal of skin moisture (a flexible GSR sensor), a body temperature (a flexible temperature sensor), and a pulse (a flexible pulse sensor);

a motion detection sensor 115 (an acceleration sensor, a gyro sensor, and a terrestrial magnetism sensor, etc.) for detecting a dynamic movement signal; and

a signal processing unit 113 configured to amplify and digitally convert signals detected by each of the sensors 115 and 120 to output the signals as sensor detection signals; and

a wireless communication unit (Bluetooth, ZigBee, etc.) 111 configured to process and transmit the digitally converted sensor detection signals according to a wireless communication standard,

wherein the micro battery 117, the biometric signal detection sensors 120, the motion detection sensor 115, the signal processing unit 113, and the wireless communication unit 111 are mounted on one FPCB module 100 to be interconnected through electrical wiring.

The micro battery 117 for supplying power may be implemented by a flexible battery or a micro battery.

The signal processing unit 113 may comprise a current to voltage converter, an amplifier, a filter unit, and an analog to digital converter.

The current to voltage converter converts a current signal provided from the motion detection sensor 115 and the biometric signal detection sensors 120 to a voltage signal, and the amplifier amplifies the voltage signal to a voltage level with a sufficient magnitude. The filter unit may have an RC filter for removing noise included in the amplified signal through the amplifier by using a band-pass filter to extract a signal corresponding to each band and for removing noise due to an external power source. The analog to digital converter converts an analog signal provided from the filter unit to a digital signal and provides the digital signal to the wireless communication unit 111.

The wireless communication unit 111 may be implemented with a short-range wireless communication standard such as Bluetooth, ZigBee, Ultra WideBand (UWB), or Wi-Fi of IEEE 802.11 series, etc. and may process data provided from the signal processing unit 113 according to a wireless communication standard and transmit a processed biometric signal to a smart device or a neighboring access point device.

As illustrated in the drawings, in an FPCB for a smart band according to a first embodiment of the present invention, the biometric signal detection sensors 120 are integrated into a flexible sensor module on the FPCB module 100, and the micro battery 117, the motion detection sensor 115, the signal processing unit 113, and the wireless communication unit 111 are integrated into a signal processing module 110 on the FPCB module 100 so that the flexible sensor module and the signal processing module 110 are physically separated.

FIG. 3 illustrates a model manufactured by integrating the flexible sensor module and the signal processing module 110 on one FPCB module 100. As illustrated in FIG. 3, when a smart band is manufactured by using an FPCB, even if a smart band wearer performs an exercise such as running or walking, a surface of the FPCB 100 may make a close contact with skin thereof, and thus, it is possible to measure a biometric signal normally.

Further, when a smart band using an FPCB according to an embodiment of the present invention is worn, various biometric signals such as a pulse, a body temperature, and skin moisture are transmitted to and displayed on a smart device such as a smart phone, and thus, a wearer can continuously monitor biometric signals such as a pulse, a body temperature, and skin moisture, autonomously adjust an exercise amount or intensity of exercise through a biometric signal monitor, as well as recognize a change of feeling or emotion such as sudden stress, and thus minimize in advance a problem according to the change of feeling and emotion.

In the foregoing embodiment, although a structure has been illustrated that mounts the integrated signal processing module 110 and the integrated flexible sensor module on one FPCB module 100, as illustrated in FIG. 2, the signal processing module 110 and the flexible sensor module may be mounted separately on different FPCBs, and thus interconnected through conductive fiber or flexible wiring.

A detailed description on the technical components illustrated in FIG. 2 which are the same as the technical components illustrated in FIG. 1 will be omitted.

An FPCB module for a smart band illustrated in FIG. 2 comprises a flexible sensor module in which biometric signal detection sensors 310, 320, and 330 for detecting at least each of skin humidity, a body temperature, and a pulse signal are mounted on a second FPCB 300;

a signal processing module in which a motion detection sensor 230 for detecting a dynamic motion signal, a signal processing unit 220 configured to amplify and digitally convert signals detected by each of the sensors 230, 310, 320, and 330 to output the signals as sensor detection signals, and a wireless communication unit 210 configured to process and transmit the digitally converted sensor detection signals according to a wireless communication standard are mounted on a first FPCB 200,

wherein the flexible sensor module and the signal processing module are interconnected through conductive fiber or flexible wiring.

Although the FPCBs for a smart band illustrated in FIG. 2 are separated to be used for the signal processing module and the flexible sensor module, because it is structured that the sensors and the component elements for signal processing are mounted on the FPCBs, each FPCB surface may make a close contact with skin of a band wearer so that, even if the band wearer exercises, biometric signals can be measured normally.

Further, when a smart band using the FPCB module illustrated in FIG. 2 is worn, various biometric signals such as a pulse, a body temperature, and skin moisture may be measured, and thus, a change of feeling and emotion may be recognized through monitoring the biometric signals such as a pulse, a body temperature, and skin moisture so that a problem according to the change of feeling and emotion can be minimized.

As described above, because the present invention can detect a body temperature and skin moisture together as a biometric signal, as well as a pulse, it is possible to monitor a change of emotion of a smart band wearer and thus to enable the wearer to cope wisely with a sudden, dangerous situation.

Furthermore, because biometric signal detection sensors are mounted on an FPCB, the sensors come into a close contact with a body of a wearer, and thus, even if the wearer walks or runs, can measure biometric signals normally, and the sensors come in close and good contact with the body, and can minimize an attachment feeling of foreign matters due to good adhesion when the smart band is worn.

Although the present invention has been described in detail hereinabove with reference to the embodiments illustrated in the drawings, which are designed to be illustrative, it should be clearly understood by a skilled person in the art that many variations and equivalents thereof can be made therefrom. Thus, the scope of the present invention should be defined only by the appended claims.

1. A flexible printed circuit board (FPCB) module for a smart band, the FPCB module comprising:
a micro battery for supplying power;
biometric signal detection sensors for detecting at least each of skin humidity, a body temperature, and a pulse signal; a motion detection sensor for detecting a dynamic movement signal;
a signal processing unit configured to amplify and digitally convert signals detected by each of the sensors to output the signals as sensor detection signals; and
a wireless communication unit configured to process and transmit the digitally converted sensor detection signals according to a wireless communication standard, wherein the micro battery, the biometric signal detection sensors, the motion detection sensor, the signal processing unit, and the wireless communication unit are mounted on the FPCB to be interconnected through electrical wiring.

2. The FPCB module of claim 1, wherein the biometric signal detection sensors are integrated into a flexible sensor module on the FPCB, and the micro battery, the motion detection sensor, the signal processing unit, and the wireless communication unit are integrated into a signal processing module on the FPCB so that the flexible sensor module and the signal processing module are physically separated.

3. A flexible printed circuit board (FPCB) module for a smart band, the FPCB module comprising:
a flexible sensor module in which biometric signal detection sensors for detecting at least each of skin humidity, a body temperature, and a pulse signal are mounted on a first FPCB; and
a signal processing module in which a movement detection sensor for detecting a dynamic movement signal, a signal processing unit configured to amplify and digitally convert signals detected by each of the sensors to output the signals as sensor detection signals, and a wireless communication unit configured to process and transmit the digitally converted sensor detection signals according to a wireless communication standard are mounted on a second FPCB,
wherein the flexible sensor module and the signal processing module are interconnected through conductive fiber or flexible wiring.
